(19) United States
(12) Patent Application Publication
(10) Pub. No.: US 2021/0059888 A1
(43) Pub. Date: Mar. 4, 2021

(54) Exoskeleton Glove

(71) Applicant: Adjuvo Motion B.V., Delft (NL)

(72) Inventors: Johannes Luijten, Delft (NL); Gijs den Butter, Delft (NL); Max Lammers, Delft (NL)

(21) Appl. No.: 17/095,609

(22) Filed: Nov. 11, 2020

Related U.S. Application Data
(63) Continuation of application No. PCT/NL2019/050286, filed on May 16, 2019.

(30) Foreign Application Priority Data
May 16, 2018 (NL) 2020941

(51) Int. Cl. A61H 1/02; G06F 3/01; B25J 9/00; B25J 13/02; B25J 9/10

(52) U.S. Cl. CPC A61H 1/0288; G06F 3/016; B25J 9/0006; B25J 13/025; B25J 19/0004; A61H 2201/1215; A61H 2201/149; A61H 2201/1638; A61H 2201/165; B25J 9/1045

(57) ABSTRACT

An exoskeleton glove (2) comprising an individual linkage mechanism (4, 5, 6, 7) for each finger, wherein the linkage mechanism comprises a series of linkages (8, 11, 15, 19) concatenated through joints (10, 17, 20), a first link-age being attached to the glove (2), and a last linkage (19) farthest from the glove being provided with a thimble (18), wherein a cable (21) guided within or alongside the linkage mechanism connects the last linkage (19) and/or the thimble (18) to the first linkage (8) so as to transfer a force through the cable (21) acting on the last linkage (19) and/or the thimble (18).

### CROSS-REFERENCE TO RELATED APPLICATIONS

This application is a continuation application of International Patent Application No. PCT/NL2019/050286, titled “Exoskeleton Glove”, filed on May 16, 2019, which claims priority to Netherlands Patent Application No. 2020941, titled “Exoskeleton Glove”, filed on May 16, 2018, and the specification and claims thereof are incorporated herein by reference.

### BACKGROUND OF THE INVENTION

### Field of the Invention

Embodiments of the present invention relate to an exoskeleton glove provided with an individual linkage mechanism for at least a finger or a thumb, wherein the linkage mechanism comprises a series of linkages concatenated through joints, a first linkage being attached to the glove, and a last linkage at a farthest end from the glove being provided with a thimble.

### Background Art

WO2018/021909 teaches an exoskeleton glove.

EP-A-2 345 951 and US2013/0219585 each disclose an exoskeleton glove provided with an individual linkage mechanism for at least a finger or a thumb, wherein the linkage mechanism comprises a series of linkages concatenated through joints, a first linkage being attached to the glove, and a last linkage at a farthest end from the glove being provided with a thimble, wherein a cable guided within or alongside the linkage mechanism connects the last linkage and/or the thimble to the first linkage so as to transfer a force through the cable acting on the last linkage and/or the thimble.

WO99/21478 discloses an exoskeleton device for measuring positions of links and angles of joints of an animate body, where the body comprises a plurality of links joined by intervening joints. The device is affixed at a first mobile terminus of said animate body, and a second fixed terminus, having device links displaced from animate links, where the device links are connected by device joints and having sensor means for measuring the angle of the device joints. Using the signals from the sensor means, one can determine the position of the terminal device link, and based on knowledge of the animate body structure calculate the animate angle joints.

Note that this application refers to a number of publications, and that due to recent publication dates certain publications are not to be considered as prior art vis-a-vis the present invention. Discussion of such publications herein is given for more complete background and is not to be construed as an admission that such publications are prior art for patentability determination purposes.

### BRIEF SUMMARY OF THE INVENTION

One objective of the present invention is to modify the known exoskeleton glove to enable a user to interact with virtual objects in a way similar to real world objects.

It is a further object of the invention to provide an exoskeleton glove which is suitable for providing force and tactile feedback to the fingers of a user of the glove.

It is still a further object of the invention to keep weight of the exoskeleton glove down and to make it possible to use the glove without features that require external powering.

In order to promote one or more of the above-mentioned objectives the exoskeleton glove of the invention has the features of one or more of the appended claims.

In a first aspect of an embodiment of the present invention, at or near the first linkage, a spring-loaded pulley is provided for winding or unwinding the cable, wherein the pulley is connected with a magnetic brake. With the cable a mechanical force feedback can be provided to the finger of the user that corresponds with the linkage mechanism. The spring-loaded pulley enables that during the motion of the finger, a continuous tension can be provided on the cable. By applying a pulley it is further realized that a linear translation is converted into a rotation for retracting the cable. The magnetic brake makes possible that at a moment when a virtual object connects to a virtual representation of the finger, the brake provides a frictional force to the pulley. This frictional force is transmitted via the cable to the tip of the finger, i.e. to the last linkage and/or the thimble.

Preferably the magnetic brake is drivable by a variable voltage. By regulating this voltage a variety of effects can be communicated to the user's finger such as the shape and the density of a virtual object.

By arranging that the glove is battery-powered, the glove will be very user-friendly without need to connect to an external power source.

The exoskeleton glove of the invention is suitably embodied such that the cable is guided over a guide wheel in the linkage mechanism which is positioned distant from the glove.

In another aspect of the invention the linkage mechanism is provided with a vibro-haptic feedback motor. With this feedback motor additional information on the virtual object can be communicated to the user's finger, such as hardness, roughness or for instance simulation of a point of entry in a virtual object. For the same purpose it is also possible that at least one actuator acting on the cable is provided.

Objects, advantages and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### BRIEF DESCRIPTION OF THE SEVERAL VIEWS OF THE DRAWINGS

The accompanying drawings, which are incorporated into and form a part of the specification, illustrate one or more embodiments of the present invention and, together with the description, serve to explain the principles of the invention. The drawings are only for the purpose of illustrating one or more embodiments of the invention and are not to be construed as limiting the invention. In the drawings:

FIG. 1A and FIG. 1B are illustrations show in different perspectives a glove of an exoskeleton glove according to the prior art provided on a hand of the user;

FIGS. 2A and 2B are illustrations showing the individual linkage mechanisms of the thumb and the fingers of the prior art glove in different perspectives;

FIG. 3 is an illustration showing a linkage mechanism for a finger of an exoskeleton glove according to an embodiment of the present invention; and

FIG. 4 is an illustration showing a sectional view of the linkage mechanism shown in FIG. 3.

### DETAILED DESCRIPTION OF THE INVENTION

Whenever in the figures the same reference numerals are applied, these numerals refer to the same parts.

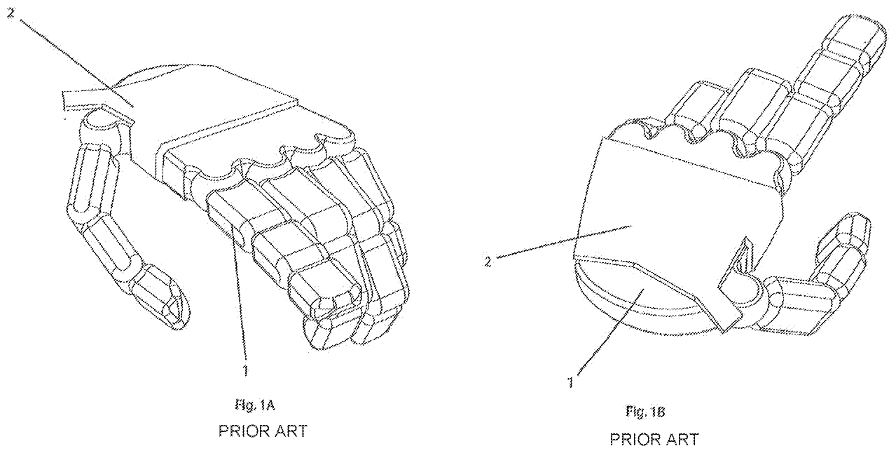

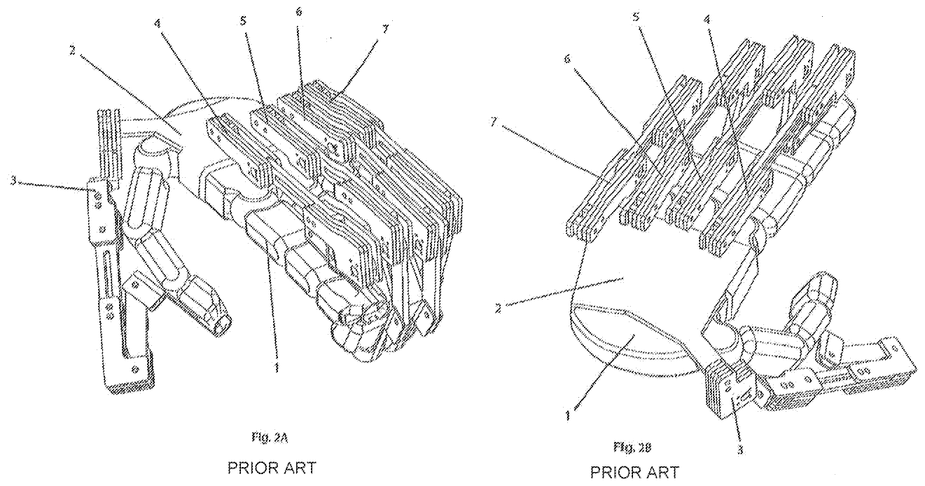

FIG. 1A and FIG. 1B show in different perspectives a user's hand 1 provided with a glove 2 of an exoskeleton glove of the prior art which is shown in more detail in FIG. 2A and FIG. 2B.

In FIG. 2A and FIG. 2B it is shown that the exoskeleton glove is provided with an individual linkage mechanism for each finger and for the thumb. It shows a linkage mechanism 3 for the thumb, and further there are a linkage mechanism 4 for the index finger, a linkage mechanism 5 for the middle finger, a linkage mechanism 6 for the ring finger, and a linkage mechanism 7 for the small finger. The linkage mechanism 3 for the thumb is a bit more complicated than the linkage mechanisms for the fingers. The construction of the linkage mechanisms for the fingers is however for each finger the same. Accordingly the linkage mechanism comprises in any case a series of linkages 8, 11, 15, 19 concatenated through joints 10, 17, 20, wherein a first linkage is attached to the glove 2, and a last linkage 19 farthest from the glove is provided with a thimble 18.

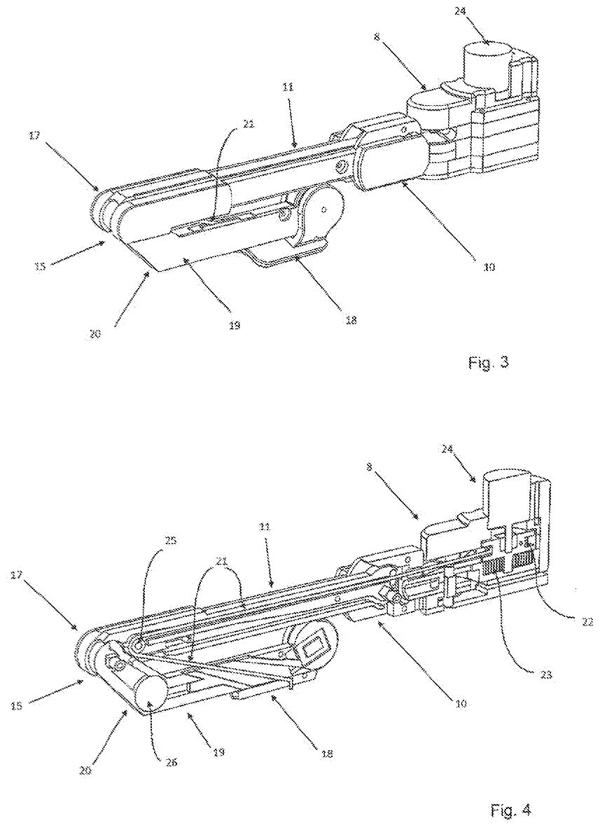

FIGS. 3 and 4 show a linkage mechanism for a single finger as forming part of the exoskeleton glove according to an embodiment of the present invention. It is noted that although not shown the linkage mechanism for the thumb of the exoskeleton glove of the embodiment of the present invention is arranged similarly, so that a separate illustration thereof is superfluous. FIG. 3 shows the linkage mechanism in full view, whereas FIG. 4 shows the linkage mechanism in a sectional view through a longitudinal axis of the mechanism.

FIGS. 3 and 4 show that the linkage mechanism comprises several sections that are interconnected with joints to enable changing their mutual angular orientation. The linkage mechanism comprises first linkage 8 which is intended to be attached to glove 2 (see FIGS. 2A and 2B), second linkage 11 connected to first linkage 8 through first joint 9, third linkage 15 connected to the second linkage 11 through second joint 17, and fourth linkage 19 connected to third linkage 15 through third joint 20, wherein fourth linkage 19 is provided with thimble 18. Thimble 18 is evidently at a farthest end from glove 2.

FIG. 3 and more clearly FIG. 4 further show that cable 21 is guided within (or alongside) the linkage mechanism that connects the last linkage 19 distant from glove 2 and/or thimble 18 to first linkage 8 so as to transfer a force through cable 21 acting on the last linkage 19 and/or thimble 18.

At or near first linkage 8, spring-loaded pulley 22 is provided for winding or unwinding cable 21. The spring for the pulley 22 is carrying reference 23.

Pulley 22 is preferably connected with magnetic brake 24, which is desirably drivable by a variable voltage, for instance by pulse width modulation PWM, which is known per se to the skilled person.

Preferably, embodiments of the present invention are battery-powered. This is not shown in the drawing but is clear for the skilled person and requires no further elucidation.

FIG. 4 further shows that cable 21 is guided over guide wheel 25 in the linkage mechanism which is positioned distant from glove 2.

Finally, FIG. 4 shows that the linkage mechanism is provided with vibro-haptic feedback motor 26.

Although the invention has been discussed in the foregoing with reference to an exemplary embodiment of a part of the exoskeleton glove of the invention, the invention is not restricted to this particular embodiment which can be varied in many ways without departing from the invention. The discussed exemplary embodiment shall therefore not be used to construe the appended claims strictly in accordance therewith. On the contrary the embodiment is merely intended to explain the wording of the appended claims without intent to limit the claims to this exemplary embodiment. The scope of protection of the invention shall therefore be construed in accordance with the appended claims only, wherein a possible ambiguity in the wording of the claims shall be resolved using this exemplary embodiment.

Embodiments of the present invention can include every combination of features that are disclosed herein independently from each other. Although the invention has been described in detail with particular reference to the disclosed embodiments, other embodiments can achieve the same results. Variations and modifications of the present invention will be obvious to those skilled in the art and it is intended to cover in the appended claims all such modifications and equivalents. The entire disclosures of all references, applications, patents, and publications cited above are hereby incorporated by reference. Unless specifically stated as being “essential” above, none of the various components or the interrelationship thereof are essential to the operation of the invention. Rather, desirable results can be achieved by substituting various components and/or reconfiguration of their relationships with one another.

1. An exoskeleton glove provided with an individual linkage mechanism for at least a finger or a thumb, wherein the linkage mechanism comprises a series of linkages concatenated through joints, a first linkage being attached to the glove, and a last linkage at a farthest end from the glove being provided with a thimble, wherein a cable guided within or alongside the linkage mechanism connects the last linkage and/or the thimble to the first linkage so as to transfer a force through the cable acting on the last linkage and/or the thimble, wherein at or near the first linkage a spring-loaded pulley is provided for winding or unwinding the cable, and wherein the pulley is connected with a magnetic brake.

2. The exoskeleton glove according to claim 1, wherein the magnetic brake is drivable by a variable voltage.

3. The exoskeleton glove according to claim 1, wherein the glove is battery-powered.

4. The exoskeleton glove according to claim 1, wherein the cable is guided over a guide wheel in the linkage mechanism which is positioned distant from the glove.

5. The exoskeleton glove according to claim 1, wherein the linkage mechanism is provided with a vibro-haptic feedback motor.

6. The exoskeleton glove according to claim 1, further comprising at least one actuator acting on the cable.
